(19) United States
(12) Patent Application Publication
(10) Pub. No.: US 2025/0339111 A1
(43) Pub. Date: Nov. 6, 2025

(54) X-RAY COMPUTED TOMOGRAPHY IMAGING APPARATUS, X-RAY HIGH-VOLTAGE APPARATUS, AND X-RAY CONTROL METHOD

(71) Applicant: Canon Medical Systems Corporation, Otawara-shi (JP)

(72) Inventor: Hidetoshi KUDO, Nasushiobara (JP)

(73) Assignee: Canon Medical Systems Corporation, Otawara-shi (JP)

(21) Appl. No.: 19/190,905

(22) Filed: Apr. 28, 2025

(30) Foreign Application Priority Data
May 2, 2024 (JP) 2024-074799

(51) Int. Cl. A61B 6/03; A61B 6/00

(52) U.S. Cl. CPC A61B 6/032; A61B 6/544

(57) ABSTRACT

An X-ray CT apparatus stores characteristic data representing a relationship between a filament current value and a tube current value in a case of switching between a first tube voltage and a second tube voltage. The apparatus sets a first tube current setting value at a time of application of the first tube voltage, and sets a second tube current setting value based on characteristic data and the first tube current setting value at a time of application of the second tube voltage. The apparatus switches between tube current feedback control based on the first tube current setting value at the time of application of the first tube voltage, and tube current feedback control based on the second tube current setting value at the time of application of the second tube voltage.

### CROSS-REFERENCE TO RELATED APPLICATION(S)

This application is based upon and claims the benefit of priority from Japanese Patent Application No. 2024-074799, filed May 2, 2024, the entire contents of which are incorporated herein by reference.

### FIELD

Embodiments described herein relate generally to an X-ray computed tomography imaging apparatus, an X-ray high-voltage apparatus, and an X-ray control method.

### BACKGROUND

Tube current modulation control is used as an X-ray computed tomography imaging scheme capable of reducing radiation to the patient. However, in the case of performing tube current modulation control in parallel with kV switching, in which high-speed switching is performed between a high tube voltage and a low tube voltage, the tube current inevitably fluctuates in accordance with the fluctuation of the tube voltage, making it difficult to perform tube current modulation control.

### BRIEF DESCRIPTION OF THE DRAWINGS

FIG. 1 is a diagram showing a configuration example of an X-ray computed tomography imaging apparatus according to the present embodiment.

FIG. 2 is a diagram showing a configuration example of an X-ray generating system including an X-ray tube and an X-ray high-voltage apparatus shown in FIG. 1.

FIG. 3 is a diagram illustrating a processing procedure in a tube current fluctuation amount measurement mode.

FIG. 4 is a graph showing a tube current output value [mA] and a tube current fluctuation amount with respect to each filament current value If[A].

FIG. 5 is a graph illustrating tube current characteristic data.

FIG. 6 is a diagram illustrating a processing procedure in tube current modulation spectrum scanning.

FIG. 7 is a diagram illustrating, as an example, tube current modulation information for reference tube voltage application (high tube voltage application).

FIG. 8 is a diagram illustrating, as an example, tube current modulation information for high tube voltage application and tube current modulation information for low tube voltage application.

FIG. 9 is a diagram showing a control sequence diagram of tube current modulation spectrum scanning.

FIG. 10 is a diagram illustrating, as an example, a display screen of a result of determination as to whether or not the tube current characteristic data needs to be re-acquired.

### DETAILED DESCRIPTION

An X-ray computed tomography imaging apparatus according to an embodiment includes an X-ray tube, a memory, tube voltage control circuitry, a setting unit, and tube current control circuitry. The X-ray tube is configured to generate X-rays. The memory is configured to store characteristic data representing a relationship between a filament current value and a tube current output value in a case of switching of a tube voltage to be applied to the X-ray tube between a first tube voltage and a second tube voltage lower than the first tube voltage. The tube voltage control circuitry is configured to switch the tube voltage to be applied to the X-ray tube between the first tube voltage and the second tube voltage. The setting unit is configured to set a tube current setting value for performing tube current modulation in parallel with the switching between the first tube voltage and the second tube voltage, in such a manner that a first tube current setting value is set at a time of application of either the first tube voltage or the second tube voltage, whichever is a reference tube voltage, and a second tube current setting value based on the characteristic data and the first tube current setting value is set at a time of application of the other one of the first tube voltage or the second tube voltage. The tube current control circuitry is configured to switch between tube current feedback control based on the first tube current setting value at the time of application of the reference tube voltage, and tube current feedback control based on the second tube current setting value at the time of application of said other tube voltage, in synchronization with the switching between the first tube voltage and the second tube voltage.

Hereinafter, an X-ray computed tomography imaging apparatus, an X-ray high-voltage apparatus, and an X-ray control method according to the present embodiment will be described in detail with reference to the drawings.

The X-ray computed tomography imaging apparatus (CT apparatus) may be of various types, including a third generation CT type and a fourth generation CT type, any of which can be applied to the present embodiment. The third generation CT type is a rotate-rotate type in which an X-ray tube and a detector integrally rotate around a subject. The fourth generation CT type is a stationary-rotate type in which only an X-ray tube rotates about a subject, with a large number of X-ray detection elements arrayed in a ring shape fixed.

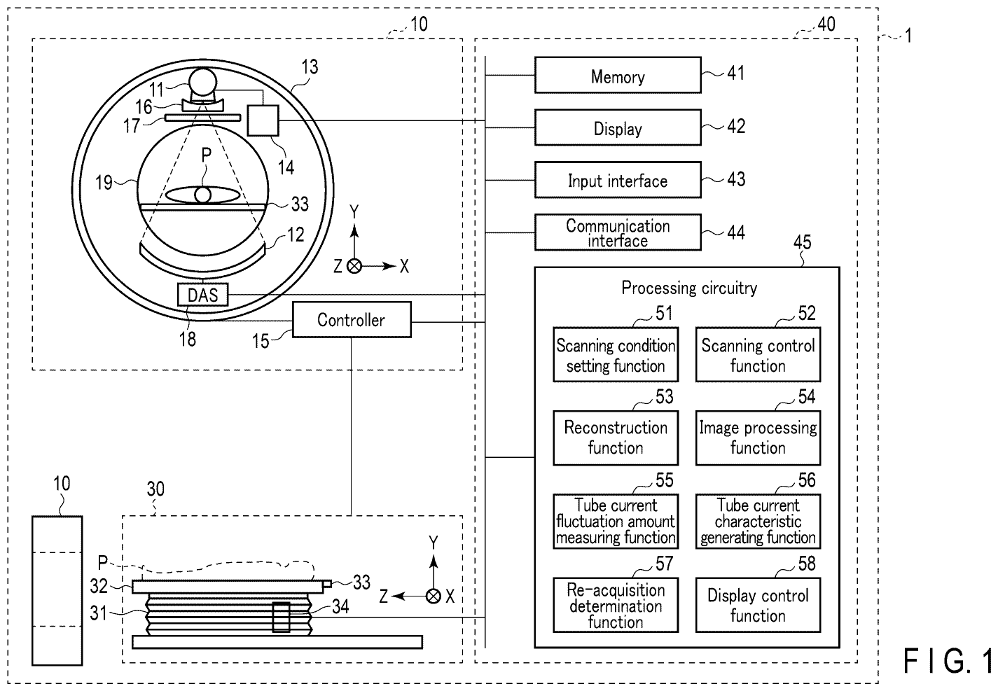

FIG. 1 is a diagram showing a configuration example of an X-ray computed tomography imaging apparatus 1 according to the present embodiment. As shown in FIG. 1, the X-ray computed tomography imaging apparatus 1 includes a gantry 10, a bed 30, and a console 40. For the sake of descriptive convenience, a plurality of gantries 10 are depicted in FIG. 1; however, the number of gantries 10 may be either one or more than one. The gantry 10 is a scan apparatus with a configuration for performing X-ray CT imaging of a subject P. The bed 30 is a transfer apparatus on which the subject P to be subjected to X-ray CT imaging is placed and which is configured to position the subject P. The console 40 is a computer configured to control the gantry 10. For example, the gantry 10 and the bed 30 are installed in a CT examination room, and the console 40 is installed in a control room adjacent to the CT examination room. The gantry 10, the bed 30, and the console 40 are communicably connected to each other wiredly or wirelessly. Note that the console 40 need not always be installed in a control room. For example, the console 40 may be installed in the same room as the gantry 10 and the bed 30. Alternatively, the console 40 may be embedded in the gantry 10.

As shown in FIG. 1, the gantry 10 includes an X-ray tube 11, an X-ray detector 12, a rotating frame 13, an X-ray high-voltage apparatus 14, a controller 15, a wedge 16, a collimator 17, and a data acquisition system (DAS) 18.

The X-ray tube 11 irradiates the subject P with X rays. More specifically, the X-ray tube 11 includes a cathode configured to generate thermoelectrons, an anode configured to generate X rays upon receiving the thermoelectrons flying from the cathode, and a vacuum tube holding the cathode and the anode. The X-ray tube 11 is connected to the X-ray high-voltage apparatus 14 via a high-voltage cable. The X-ray high-voltage apparatus 14 applies a tube voltage between the cathode and the anode. Through the application of the tube voltage, thermoelectrons fly from the cathode to the anode. With the thermoelectrons flying from the cathode to the anode, a tube current flows. As a result of the thermoelectrons colliding with the anode, X rays are generated.

The X-ray detector 12 detects the X rays emitted from the X-ray tube 11 and transmitted through the subject P, and outputs an electrical signal corresponding to the dose of the detected X rays to the DAS 18. The X-ray detector 12 has a structure in which a plurality of strings of X-ray detection elements, each with a plurality of X-ray detection elements aligned in a channel direction, are aligned in a slice direction (column direction). The X-ray detector 12 is, for example, an indirect-conversion-type detector including a grid, a scintillator array, and an optical sensor array. The scintillator array includes a plurality of scintillators. The scintillator outputs an amount of light corresponding to the dose of incident X rays. The grid includes an X-ray shielding plate arranged on an X-ray incident surface side of the scintillator array and configured to absorb scattered X rays. Note that the grid is also referred to as a collimator (a one-dimensional or two-dimensional collimator). The optical sensor array converts light from each scintillator into an electrical signal corresponding to the amount of light. Examples of the optical sensor that may be used include a photodiode. Note that the X-ray detector 12 may be a direct-conversion-type detector.

The rotating frame 13 is an annular frame configured to support the X-ray tube 11 and the X-ray detector 12 so as to allow them to rotate about a rotation axis (a Z axis). Specifically, the rotating frame 13 supports the X-ray tube 11 and the X-ray detector 12 so as to make them face each other. The rotating frame 13 is supported on a fixed frame (not shown) so as to be rotatable about the rotation axis. The controller 15 rotates the rotating frame 13 about the rotation axis to rotate the X-ray tube 11 and the X-ray detector 12 about the rotation axis. The rotating frame 13 rotates about the rotation axis at a predetermined angular velocity upon receiving drive power from the drive mechanism of the controller 15. A field of view (FOV) is set in an opening portion 19 of the rotating frame 13.

In the present embodiment, a rotation axis of the rotating frame 13 in a non-tilted state or a longitudinal direction of a top plate 33 of the bed 30 is defined as a “Z-axis direction”, an axis direction that is orthogonal to the Z-axis direction and is horizontal to the floor surface is defined as an “X-axis direction”, and an axis direction that is orthogonal to the Z-axis direction and is vertical to the floor surface is defined as a “Y-axis direction”.

The X-ray high-voltage apparatus 14 includes a high-voltage generator and an X-ray controller. The high-voltage generator includes electrical circuitry such as a transformer and a rectifier, and generates a high voltage to be applied to the X-ray tube 11 and a filament current to be supplied to the X-ray tube 11. The X-ray controller controls an output voltage corresponding to the X rays emitted by the X-ray tube 11. The high-voltage generator may be either a transformer type or an inverter type. The X-ray high-voltage apparatus 14 may be provided on the rotating frame 13 in the gantry 10 or provided on a fixed frame (not shown) in the gantry 10.

The wedge 16 adjusts the dose of X rays applied to the subject P. Specifically, the wedge 16 attenuates X rays such that the dose of X rays applied from the X-ray tube 11 to the subject P has a predetermined distribution. Examples of the wedge 16 that may be used include a metal plate made of aluminum, such as a wedge filter or bow-tie filter.

The collimator 17 limits an irradiation range of X rays transmitted through the wedge 16. The collimator 17 slidably supports a plurality of lead plates for shielding X rays and adjusts the form of the slit formed by the lead plates. Note that the collimator 17 is also referred to as an “X-ray aperture”.

The DAS 18 reads, from the X-ray detector 12, an electrical signal corresponding to the dose of X rays detected by the X-ray detector 12. The DAS 18 amplifies the read electrical signal and acquires detection data having a digital value corresponding to the dose of X rays over the duration of a view period by integrating the electrical signal throughout the view period. Detection data is referred to as “projection data”. The DAS 18 is implemented by, for example, an application-specific integrated circuit (ASIC) provided with a circuitry element capable of generating projection data. The projection data is transmitted to the console 40 via a non-contact data transmitter or the like.

An integral X-ray detector 12 and an X-ray computed tomography imaging apparatus 1 provided with the integral X-ray detector 12 will be described as an example; however, the technique according to the present embodiment is also applicable to a photon counting X-ray detector.

The controller 15 controls the X-ray high-voltage apparatus 14 and the DAS 18 to execute X-ray CT imaging in accordance with a scanning control function 52 of processing circuitry 45 of the console 40. The controller 15 includes processing circuitry with a central processing unit (CPU), a microprocessor unit (MPU), or the like, and a drive mechanism such as a motor and an actuator. The processing circuitry includes, as hardware resources, a processor such as a CPU and memories such as a read-only memory (ROM) and a random-access memory (RAM). The controller 15 executes various types of functions with a processor configured to execute programs expanded in a memory. Note that the respective types of functions need not always be implemented by a single processing circuit. Processing circuitry may be formed by combining a plurality of independent processors, with each processor configured to execute a corresponding function by executing a corresponding program. The controller 15 may be implemented by an ASIC or a field-programmable gate array (FPGA). Alternatively, the controller 15 may be implemented by another complex programmable logic device (CPLD) or a simple programmable logic device (SPLD). The controller 15 has a function of controlling the operations of the gantry 10 and the bed 30 upon receiving input signals from an input interface 43 (to be described later) attached to the console 40 or the gantry 10. For example, the controller 15 performs control to rotate the rotating frame 13, to tilt the gantry 10, and to operate the bed 30 and the top plate 33 upon receiving input signals. Note that the controller 15 implements the control to tilt the gantry 10 by rotating the rotating frame 13 about an axis parallel to the X-axis direction in accordance with tilt angle information input by the input interface attached to the gantry 10. Note that the controller 15 may be provided on the gantry 10 or the console 40.

The bed 30 includes a base 31, a support frame 32, the top plate 33, and a bed drive device 34. The base 31 is installed on the floor surface. The base 31 is a housing configured to support the support frame 32 so as to allow it to move in the vertical direction (Y-axis direction) with respect to the floor surface. The support frame 32 is a frame provided on an upper portion of the base 31. The support frame 32 supports the top plate 33 so as to allow it to slide along the rotation axis (Z axis). The top plate 33 is a flexible plate on which the subject P is placed.

The bed drive device 34 is accommodated in the housing of the bed 30. The bed drive device 34 is a motor or actuator that generates drive power for moving the support frame 32 and the top plate 33 on which the subject P is placed. The bed drive device 34 operates under the control of the console 40 and the like.

The console 40 includes a memory 41, a display 42, the input interface 43, a communication interface 44, and the processing circuitry 45. Data communication is performed among the memory 41, the display 42, the input interface 43, the communication interface 44, and the processing circuitry 45 via a bus (BUS). Although the console 40 will be described as being separate from the gantry 10, the gantry 10 may include the console 40 or part of the constituent elements of the console 40.

The memory 41 is a storage device such as a hard disk drive (HDD), a solid-state drive (SSD), or an integrated circuit storage device, which stores various types of information. The memory 41 may be a portable storage medium such as a compact disc (CD), a digital versatile disc (DVD), a Blu-ray (registered trademark) Disc (BD), a flash memory, etc., as well as an HDD, an SSD, etc. The memory 41 may be a drive device configured to read and write various types of information between semiconductor memory elements such as a flash memory and a RAM. In addition, the save area of the memory 41 may be located in the X-ray computed tomography imaging apparatus 1 or in an external storage device connected via a network. The memory 41 stores, for example, projection data and reconstruction image data.

The display 42 displays various types of information. For example, the display 42 outputs a CT image generated by the processing circuitry 45, a graphical user interface (GUI) for accepting various types of operations from the operator, and the like. As the display 42, any display of various types can be suitably used. Examples of the display 42 that may be used include a liquid-crystal display (LCD), a cathode-ray tube (CRT), an organic electroluminescence display (OELD), and a plasma display.

Note that the display 42 may be provided in any location in the control room. The display 42 may be provided on the gantry 10. The display 42 may be of a desktop type or may be configured of a tablet terminal or the like wirelessly communicable with the main body of the console 40. As the display 42, either one or more than one projectors may be used.

The input interface 43 accepts various types of input operations from the operator, converts the accepted input operations into electrical signals, and outputs them to the processing circuitry 45. For example, the input interface 43 accepts, from the operator, acquisition conditions for acquiring projection data, reconstruction conditions for reconstructing a CT image, image processing conditions for generating a post-processing image from the CT image, and the like. Examples of the input interface 43 that may be used include a mouse, a keyboard, a trackball, switches, buttons, a joystick, a touch pad, a touch panel display, and the like. Note that, in the present embodiment, the input interface 43 is not limited to one that includes physical operation components such as a mouse, a keyboard, a trackball, switches, buttons, a joystick, a touch pad, and a touch panel display. Examples of the input interface 43 include electrical signal processing circuitry configured to accept an electrical signal corresponding to an input operation from an external input device provided separately from the apparatus and to output the electrical signal to the processing circuitry 45. The input interface 43 may be provided on the gantry 10. The input interface 43 may be configured of a tablet terminal or the like capable of wirelessly communicating with the main body of the console 40.

The communication interface 44 includes a network interface card (NIC) for communicating various types of data with an external device such as a workstation, a picture archiving and communication system (PACS), a radiological information system (RIS), or a hospital information system (HIS) via a network.

The processing circuitry 45 controls an overall operation of the X-ray computed tomography imaging apparatus 1 in accordance with an electrical signal corresponding to an input operation output from the input interface 43. The processing circuitry 45 generates image data based on the electrical signal output from the X-ray detector 12. For example, the processing circuitry 45 includes, as hardware resources, a processor such as a CPU, an MPU, or a GPU, as well as memories such as a ROM and a RAM. The processing circuitry 45 executes a scanning condition setting function 51, the scanning control function 52, a reconstruction function 53, an image processing function 54, a tube current fluctuation amount measuring function 55, a tube current characteristic generating function 56, a re-acquisition determination function 57, a display control function 58, and the like through a processor configured to execute programs expanded in the memory.

Note that the functions 51 to 58 need not always be implemented by a single processing circuit. Processing circuitry may be configured by combining a plurality of independent processors, with each processor configured to implement a corresponding one of the functions 51 to 58 by executing the corresponding program.

With the scanning condition setting function 51, the processing circuitry 45 sets various scanning conditions. It is assumed that scanning in the present embodiment is spectrum scanning (hereinafter referred to as “tube current modulation spectrum scanning”) in which tube current modulation is performed in parallel with switching between a first tube voltage and a second tube voltage lower than the first tube voltage. As the scanning conditions, a setting value of the first tube voltage, a setting value of the second tube voltage, a temporal change in the setting value of the first tube current at the time of application of the first tube voltage (hereinafter referred to as “first tube current modulation information”), a temporal change in the setting value of the second tube current at the time of application of the second tube voltage (hereinafter referred to as “second tube current modulation information”), a rotation speed of the rotating frame 13, and the like are set. The processing circuitry 45 generates first tube current modulation information, which indicates a temporal change in a first tube current setting value for periodically modulating the tube current. Also, the processing circuitry 45 generates second tube current modulation information, which indicates a temporal change in a second tube current setting value that varies according to the periodic modulation of the tube current, based on the first tube current modulation information and the tube current characteristic data. The tube current characteristic data refers to data representing a relationship between a filament current value and a tube current output value at the time of switching of the tube voltage to be applied to the X-ray tube 11 between the first tube voltage and the second tube voltage.

With the scanning control function 52, the processing circuitry 45 controls the X-ray high-voltage apparatus 14, the controller 15, and the DAS 18 in accordance with the scanning conditions set by the scanning condition setting function 51, and executes tube current modulation spectrum scanning. At this time, the X-ray high-voltage apparatus 14 switches between tube current feedback control based on the first tube current setting value at the time of application of either the first tube voltage or the second tube voltage whichever is a reference tube voltage, and tube current feedback control based on the second tube current setting value at the time of application of the other tube voltage, in synchronization with the switching between the first tube voltage and the second tube voltage.

With the reconstruction function 53, the processing circuitry 45 subjects the projection data output from the DAS 18 to preprocessing such as logarithmic conversion processing, offset correction processing, inter-channel sensitivity correction processing, beam hardening correction, and interpolation processing in the event of data loss as a result of the tube voltage switching. The processing circuitry 45 generates a CT image (hereinafter referred to as a “reference material image”) by subjecting the preprocessed projection data to material discrimination and performing reconstruction processing on the projection data subjected to the material discrimination. As the reconstruction processing, the filtered back projection method, the iterative reconstruction method, and machine-learning-based reconstruction processing can be used. Note that the processing circuitry 45 may generate a CT image (also referred to as an “integral image”) by performing reconstruction processing on projection data not subjected to material discrimination.

With the image processing function 54, the processing circuitry 45 converts the CT image generated by the reconstruction function 53 into a section image of a given section or a rendering image in a given viewpoint direction. The conversion is performed based on an input operation accepted from the operator via the input interface 43. For example, the processing circuitry 45 generates a rendering image in a given viewpoint direction by subjecting the CT image to three-dimensional image processing such as volume rendering, surface volume rendering, image value projection processing, multiplanar reconstruction (MPR) processing, or curved-planar reconstruction (MPR). Note that generation of a rendering image in a given viewpoint direction may be directly performed by the reconstruction function 53.

With the tube current fluctuation amount measuring function 55, the processing circuitry 45 controls the X-ray high-voltage apparatus 14 and the controller 15 to execute a tube current fluctuation amount measurement mode. The tube current fluctuation amount measurement mode differs from X-ray CT scanning of the subject P, and is a mode for measuring a tube current fluctuation amount. In the tube current fluctuation amount measurement mode, the processing circuitry 45 measures, for each filament current value, a tube current output value of the X-ray tube 11 and a tube current fluctuation amount representing a fluctuation amount of the tube current output value, at the time of switching the tube voltage between a first tube voltage and a second tube voltage lower than the first tube voltage, with the filament current value fixed.

With the tube current characteristic generating function 56, the processing circuitry 45 generates, based on the filament current values, the tube current output value, and the tube current fluctuation amount obtained through the tube current fluctuation amount measuring function 55, tube current characteristic data for the combination of the first tube voltage and the second tube voltage. The tube current characteristic data is stored in the memory 41 or the like.

With the re-acquisition determination function 57, the processing circuitry 45 determines whether or not the tube current characteristic data needs to be re-acquired, based on an amount of fluctuation of the filament current value in accordance with the switching between the first tube voltage and the second tube voltage. If it has been determined that the tube current characteristic data needs to be re-acquired, the tube current characteristic data is re-acquired through the tube current fluctuation amount measuring function 55, and the tube current characteristic generating function 56. If it has been determined that the tube current characteristic data does not need to be re-acquired, the current tube current characteristic data continues to be used.

With the display control function 58, the processing circuitry 45 displays various types of information on the display 42. In an example, the processing circuitry 45 displays various types of images generated by the image processing function 54 on the display 42. In another example, the processing circuitry 45 displays determination results of the re-acquisition determination function 57.

Although the console 40 has been described as a single console configured to execute a plurality of functions, different consoles may be provided to execute the respective functions. The processing circuitry 45 need not always be included in the console 40 and may be included in a comprehensive server configured to comprehensively perform processing for projection data acquired by a plurality of medical image diagnosis apparatuses. Postprocessing may be performed by either the console 40 or an external workstation. In addition, the console 40 and the workstation may concurrently perform postprocessing.

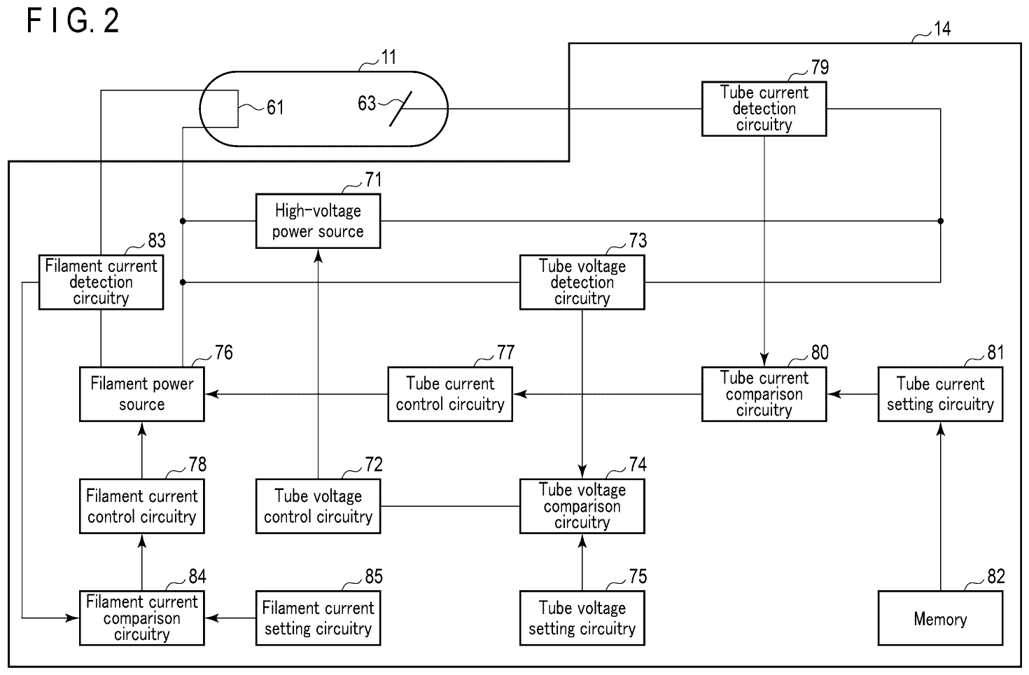

FIG. 2 is a diagram showing a configuration example of an X-ray generating system including the X-ray tube 11 and the X-ray high-voltage apparatus 14 shown in FIG. 1. As shown in FIG. 2, the X-ray tube 11 includes a cathode 61 and an anode 63. The cathode 61 includes, for example, a filament formed of metal such as tungsten. The cathode 61 is connected to the X-ray high-voltage apparatus 14 via a cable or the like. The cathode 61 generates heat and emits thermoelectrons upon receiving a supply of a filament current and an application of a cathode voltage from the X-ray high-voltage apparatus 14. The anode 63 is a disk-shaped electrode formed of a heavy metal such as tungsten or molybdenum. The anode 63 rotates in accordance with the rotation of the rotor (not shown) about the axis. The X-ray high-voltage apparatus 14 applies a high tube voltage between the cathode 61 and the anode 63. The thermoelectrons emitted from the cathode 61 collide with the anode 63 by the action of the tube voltage. The anode 63 generates X rays upon receiving the thermoelectrons.

As shown in FIG. 2, the X-ray high-voltage apparatus 14 includes a high-voltage power source 71, tube voltage control circuitry 72, tube voltage detection circuitry 73, tube voltage comparison circuitry 74, tube voltage setting circuitry 75, a filament power source 76, tube current control circuitry 77, filament current control circuitry 78, tube current detection circuitry 79, tube current comparison circuitry 80, tube current setting circuitry 81, a memory 82, filament current detection circuitry 83, filament current comparison circuitry 84, and filament current setting circuitry 85. The circuitry of the X-ray high-voltage apparatus 14 is realized by, for example, an ASIC or an FPGA.

The high-voltage power source 71 generates a tube voltage to be applied to the X-ray tube 11 in accordance with control of the tube voltage control circuitry 72. In the case of an inverter-type X-ray high-voltage apparatus, for example, the high-voltage power source 71 includes an AC/DC converter configured to convert an AC voltage from a commercial power supply into a DC voltage, an inverter configured to convert the DC voltage from the AC/DC converter into an AC voltage, a transformer configured to step up the AC voltage from the inverter, and high-voltage rectifying and smoothing circuitry configured to generate a high DC voltage by rectifying and smoothing the AC voltage stepped up by the transformer. The high DC voltage from the high-voltage rectifying/smoothing circuitry is applied as a tube voltage between the cathode 61 and the anode 63 of the X-ray tube 11.

The tube voltage detection circuitry 73 detects the voltage applied between the cathode 61 and the anode 63 as a tube voltage value. A signal (hereinafter referred to as a “tube voltage output signal”) of the detected tube voltage value (hereinafter referred to as a “tube voltage output value”) is supplied to the tube voltage comparison circuitry 74.

The tube voltage setting circuitry 75 switches a setting value of the tube voltage in synchronization with the switching between the first tube voltage and the second tube voltage. The tube voltage setting circuitry 75 sets the tube voltage to a first tube voltage setting value for application of the first tube voltage, and sets the tube voltage to a second tube voltage setting value for application of the second tube voltage. The first tube voltage setting value and the second tube voltage setting value are set by the scanning condition setting function 51. A signal (hereinafter referred to as a “tube voltage setting signal”) indicating a tube voltage setting value (hereinafter referred to as a “tube voltage setting value”) is supplied to the tube voltage comparison circuitry 74.

The tube voltage comparison circuitry 74 inputs the tube voltage setting signal from the tube voltage setting circuitry 75 and the tube voltage output signal from the tube voltage detection circuitry 73, and subtracts the tube voltage output signal from the tube voltage setting signal, thereby generating a signal (hereinafter referred to as “differential voltage signal”) indicating a differential value between the tube voltage setting value and the tube voltage output value. The differential voltage signal is supplied to the tube voltage control circuitry 72.

In tube current modulation spectrum scanning, the tube voltage control circuitry 72 switches the tube voltage to be applied to the X-ray tube 11 between the first tube voltage and the second tube voltage. An operation of the switching between the first tube voltage and the second tube voltage is referred to as “kV switching”. Specifically, the tube voltage control circuitry 72 inputs a synchronization signal to instruct a timing of switching the tube voltage from the controller 15, and controls, at the timing instructed by the synchronization signal, the high-voltage power source 71 by comparison between the tube voltage output value and the tube voltage setting value, namely, based on the differential voltage signal. More specifically, the tube voltage control circuitry 72 performs feedback control (hereinafter referred to as “tube voltage feedback control”) of the high-voltage power source 71 in such a manner that the tube voltage output value converges to the tube voltage setting value.

The filament power source 76 generates a filament current for heating the filament of the cathode 61. Specifically, the filament power source 76 includes inverter circuitry configured to control a current to be applied to the filament of the cathode 61. The filament power source 76 performs, in the tube current modulation spectrum scanning, feedback control (hereinafter referred to as “tube current feedback control”) using a tube current in accordance with control of the tube current control circuitry 77, and performs, in the tube current fluctuation amount measurement mode, feedback control using the filament current (hereinafter referred to as “filament current feedback control”) in accordance with control of the filament current control circuitry 78.

The tube current detection circuitry 79 is connected between the high-voltage power source 71 and the X-ray tube 11. The tube current detection circuitry 79 detects, as a tube current value, a current that has flown as a result of thermoelectrons flowing from the cathode 61 to the anode 63. A signal (hereinafter referred to as a “tube current output signal”) of the detected tube current value (hereinafter referred to as a “tube current output value”) is supplied to the tube current comparison circuitry 80.

The tube current setting circuitry 81 sets a tube current setting value for performing tube current modulation in parallel with the switching between the first tube voltage and the second tube voltage. Specifically, the tube current setting circuitry 81 sets a first tube current setting value at the time of application of either the first tube voltage or the second tube voltage whichever is a reference tube voltage, and sets a second tube current setting value based on tube current characteristic data and the first tube current setting value at the time of application of the other tube voltage. The tube current setting circuitry 81 sets the first tube current setting value based on the first tube current modulation information, and sets the second tube current setting value based on the second tube current modulation information. More specifically, the tube current setting circuitry 81 sets a present value of the first tube current modulation information at the time of application of the reference tube voltage as the first tube current setting value, and sets a present value of the second tube current modulation information at the time of application of the other tube voltage as the second tube current setting value. The first tube current modulation information, the second tube current modulation information, and the tube current characteristic data are stored in the memory 82. A signal indicating a tube current setting value (hereinafter referred to as a “tube current setting signal”) is supplied to the tube current comparison circuitry 80.

The tube current comparison circuitry 80 inputs the tube current setting signal from the tube current setting circuitry 81 and a tube current output signal from the tube current detection circuitry 79. The tube current comparison circuitry 80 generates a signal (hereinafter referred to as a “differential tube current signal”) indicating a differential value between the tube current setting value and the tube current output value by subtracting the tube current output signal from the tube current setting signal. Specifically, the tube current comparison circuitry 80 generates a differential tube current signal indicating a differential value obtained by subtracting the tube current output value from the first tube current setting value at the time of application of the reference tube voltage, and generates a differential tube current signal indicating a differential value obtained by subtracting the tube current output value from the second tube current setting value at the time of application of the other tube voltage. The differential tube current signal is supplied to the tube current control circuitry 77.

In the tube current modulation spectrum scanning, the tube current control circuitry 77 performs tube current feedback control via the filament power source 76. Specifically, the tube current control circuitry 77 switches between tube current feedback control based on the first tube current setting value at the time of application of the reference tube voltage, and tube current feedback control based on the second tube current setting value at the time of application of the other tube voltage, in synchronization with the switching between the first tube voltage and the second tube voltage. More specifically, the tube current control circuitry 77 controls the tube current by controlling the filament current generated by the filament power source 76 in accordance with a differential tube current signal from the tube current comparison circuitry 80, in such a manner that a differential tube current signal becomes zero, in other words, the tube current output value converges to the tube current setting value.

The filament current detection circuitry 83 is connected between the filament power source 76 and the X-ray tube 11. The filament current detection circuitry 83 detects an output current of the filament power source, and outputs its effective value as a detection value. The effective value of the current for heating the filament of the cathode 61 is detected as a filament current value. A signal (hereinafter referred to as a “filament current output signal”) of the detected filament current value (hereinafter referred to as a “filament current output value”) is supplied to the filament current comparison circuitry 84.

The filament current setting circuitry 85 sets a plurality of filament setting values to be used in the tube current fluctuation amount measurement mode. A signal (hereinafter referred to as a “filament current setting signal”) indicating the filament current setting value is supplied to the filament current comparison circuitry 84.

The filament current comparison circuitry 84 inputs the filament current setting signal from the filament current setting circuitry 85 and the filament current output signal from the filament current detection circuitry 83. The filament current comparison circuitry 84 generates a signal (hereinafter referred to as a “differential filament current signal”) indicating a differential value between the filament current setting value and the filament current detection value by subtracting the filament current output signal from the filament current setting signal. The differential filament current signal is supplied to the filament current control circuitry 78.

In the tube current fluctuation amount measurement mode, the filament current control circuitry 78 performs filament current feedback control via the filament power source 76. Specifically, at the time of kV switching in which the tube voltage is switched between the first tube voltage and the second tube voltage, the filament current control circuitry 78 controls the filament current in such a manner that the filament current detection value converges to a target value of a plurality of filament current setting values, in other words, the differential filament current signal from the filament current comparison circuitry 84 becomes zero.

The memory 82 is a storage device such as a hard disk drive (HDD), a solid-state drive (SSD), or an integrated circuit storage device, which stores various types of information. The memory 82 may be a drive device configured to read and write various types of information from and to semiconductor memory elements such as a CD, a DVD, a BD, a flash memory, or a RAM. In addition, the save area of the memory 82 may be located in the X-ray high-voltage apparatus 14, in the memory 41 of the console 40, or in an external storage device connected via a network.

The circuits of the X-ray high-voltage apparatus 14 are not limited to an ASIC or an FPGA, and may be realized by a CPLD or an SPLD, or by a processor such as a CPU or a memory such as a ROM and a RAM. The circuits included in the X-ray high-voltage apparatus 14 may be configured of a single circuit, or may be configured of different circuits. Also, the circuits of the X-ray high-voltage apparatus 14 may be configured of a single circuit, or may be configured of a combination of a plurality of independent circuits.

Hereinafter, an operation example of the X-ray computed tomography imaging apparatus 1 according to the present embodiment will be described.

The X-ray computed tomography imaging apparatus 1 according to the present embodiment executes a tube current fluctuation amount measurement mode to measure a tube current fluctuation amount and generate tube current characteristic data, and then execute tube current modulation spectrum scanning using the generated tube current characteristic data. First, a processing procedure in the tube current fluctuation amount measurement mode will be described. In the description that follows, the first tube voltage is referred to as a “high tube voltage” and the second tube voltage is referred to as a “low tube voltage”. Although the high tube voltage value and the low tube voltage value may be set to any value, a value from 40 kV to 160 kV is arbitrarily selected herein as an example.

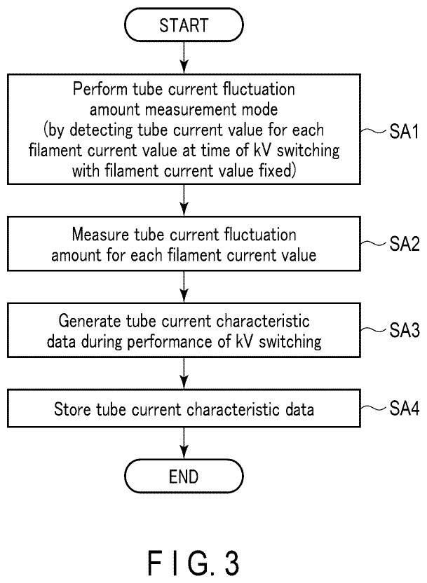

FIG. 3 is a diagram illustrating a processing procedure in a tube current fluctuation amount measurement mode. As shown in FIG. 3, with the tube current fluctuation amount measuring function 55, the processing circuitry 45 implements a tube current fluctuation amount measurement mode by controlling the X-ray high-voltage apparatus 14 and the controller 15 (step SA1). At step SA1, the processing circuitry 45 measures, for each filament current value, a tube current output value of the X-ray tube 11 at the time of alternately switching the tube voltage between a high tube voltage and a low tube voltage with the filament current value fixed. In the tube current fluctuation amount measurement mode, the subject P does not need to be placed on the bed 30. After step SA1, with the tube current fluctuation amount measuring function 55, the processing circuitry 45 measures a tube current fluctuation amount for each of the filament current values (step SA2).

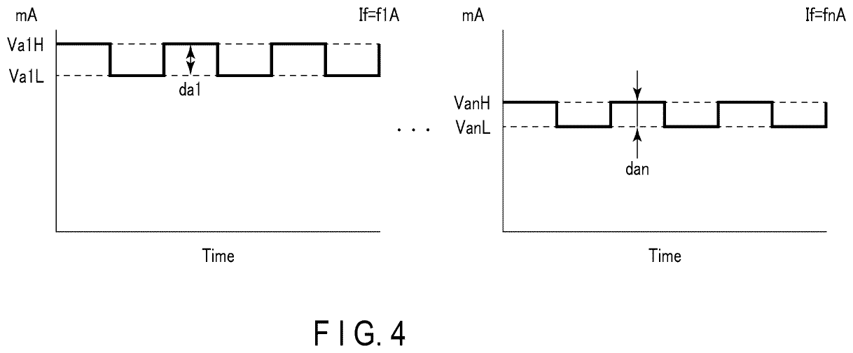

FIG. 4 is a graph showing a tube current output value mA and a tube current fluctuation amount with respect to each filament current value If[A]. In the graph shown in FIG. 4, the vertical axis denotes a tube current value mA, and the lateral axis denotes time. In the tube current fluctuation amount measurement mode, a given number n is specified as the filament current value If. The number n is set to a number with which a data amount capable of generating a tube current characteristic diagram can be generated. A given value is set as the filament current value If from a range of values that can be actually taken in the tube current modulation spectrum scanning.

It is assumed herein that the filament current value If is set to f1, as shown in the left diagram of FIG. 4. The X-ray high-voltage apparatus 14 performs kV switching of alternately switching the tube voltage between a high tube voltage and a low tube voltage, with the filament current value If fixed to f1, and measures and records a tube current output value at the time of application of the high tube voltage and a tube current output value at the time of application of the low tube voltage. Each of the high tube voltage value and the low tube voltage value are set to a given value that may be actually adopted in the tube current modulation spectrum scanning. The high tube voltage value and the low tube voltage value are not particularly limited to specific values; however, it is assumed herein that the high tube voltage value is 140 kV and that the low tube voltage value is 80 kV.

The filament current control circuitry 78 performs, in parallel with the kV switching, filament current feedback control of the filament power source 76 in such a manner that the filament current output value detected by the filament current detection circuitry 83 maintains the filament current value f1. During the performance of the kV switching and the filament current feedback, the tube current detection circuitry 79 detects a tube current value. It is assumed that a tube current value Va1H has been detected at the time of application of the high tube voltage, and that a tube current value Va1L has been detected at the time of application of the low tube voltage. The detected tube current value is recorded in the memory 41, etc. Upon detecting the tube current value, the processing circuitry 45 calculates a tube current fluctuation amount da1 representing a difference between the tube current value Va1H at the time of application of the high tube voltage and the tube current value Va1L at the time of application of the low tube voltage.

It can be expected that, in actuality, the tube current value Va1H and the tube current value Va1L fluctuate according to time. During a period soon after the start of the supply of the filament current, in particular, there is a tendency for the temperature of the filament to become unstable, causing the tube current value to fluctuate. It is therefore preferable that the processing circuitry 45 measure the tube current output value and the tube current fluctuation amount in a period of stabilization of the temperature of the filament. More specifically, it is preferable that statistical values such as a mean and an intermediate of the tube current output value and the tube current fluctuation amount in the period of stabilization be measured. It is preferable that the period of stabilization of the temperature of the filament be set to a period after a predetermined period of time has passed from the starting time of supplying of the filament current.

Measurement of the tube current value and the tube current fluctuation amount is performed with respect to each of the n filament current values. A combination of the tube current value and the tube current fluctuation amount is stored in the memory 41 in association with the filament current value. Also, the combination of the tube current value and the tube current fluctuation amount is measured and stored for each expected combination of the high tube voltage value and the low tube voltage value.

After step SA2, the processing circuitry 45 generates tube current characteristic data during the performance of the kV switching, through the tube current characteristic generating function 56 (step SA3). At step SA3, the processing circuitry 45 generates tube current characteristic data for the combination of the high tube voltage value and the low tube voltage value based on a plurality of filament current values and the tube current output value and the tube current fluctuation amount for each of the filament current values.

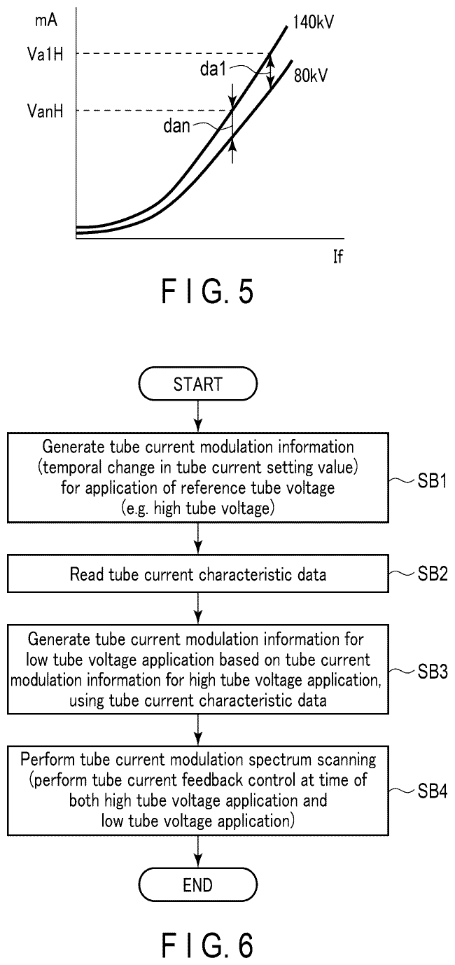

FIG. 5 is a graph illustrating tube current characteristic data. In the graph shown in FIG. 5, the vertical axis denotes a tube current value mA, and the lateral axis denotes a filament current value If. The processing circuitry 45 plots a tube current value at the time of application of a high tube voltage and a tube current value at the time of application of a low tube voltage, which are stored in the memory 41, on the graph for each filament current value. A filament current value that has not been measured and a tube current value corresponding thereto can be interpolated or extrapolated from the measured filament current value and the tube current value corresponding thereto. Thereby, tube current characteristic data is generated. Note that a curve representing a relationship between the tube current value and the filament current value in the case of a constant filament current value will be referred to as a “tube current characteristic curve”.

The dominant factor affecting the tube current is the temperature of the filament. Accordingly, it does not necessarily mean that a tube current output value identical to the tube current setting value can be obtained by supplying a filament current of a current value (filament current setting value) corresponding to the tube current setting value to the filament. As described above, the tube current characteristic data is generated based on a tube current value obtained in the filament heated by actually performing, in the X-ray tube 11 used for tube current modulation spectrum scanning, kV switching between a high tube voltage value and a low tube voltage value that may be actually adopted. Accordingly, the tube current characteristic data represents a fluctuation of the tube current value at the filament temperature at the time of supplying of a filament current with each filament current value in the case where kV switching between a high tube voltage value and a low tube voltage value is performed. By employing such tube current characteristic data unique to the X-ray tube 11, it can be seen, for example, that the tube current at the time of application of a low tube voltage (80 kV) at the filament temperature at which the tube current of Va1H flows at the time of application of a high tube voltage (140 kV) is Va1H−da1=Va1L.

After step SA3, the processing circuitry 45 stores tube current characteristic data generated at step SA3 (step SA4). The tube current characteristic data is stored in the memory 41 or the like for each combination of the high tube voltage value and the low tube voltage value.

With the foregoing, processing of the tube current fluctuation amount measurement mode ends.

Next, a processing procedure in tube current modulation spectrum scanning will be described.

FIG. 6 is a diagram illustrating a processing procedure in tube current modulation spectrum scanning. Note that scanning conditions such as a high tube voltage value and a low tube voltage value used in tube current modulation spectrum scanning are separately set.

As shown in FIG. 6, with the scanning condition setting function 51, the processing circuitry 45 generates tube current modulation information for reference tube voltage application (step SB1). The reference tube voltage refers to either a high tube voltage or a low tube voltage whichever is a reference tube voltage for the tube current modulation. Specifically, it refers to a tube voltage at which tube current modulation information is generated based on a subject body thickness. In the description that follows, it is assumed that the reference tube voltage is a high tube voltage, and that the other tube voltage is a low tube voltage.

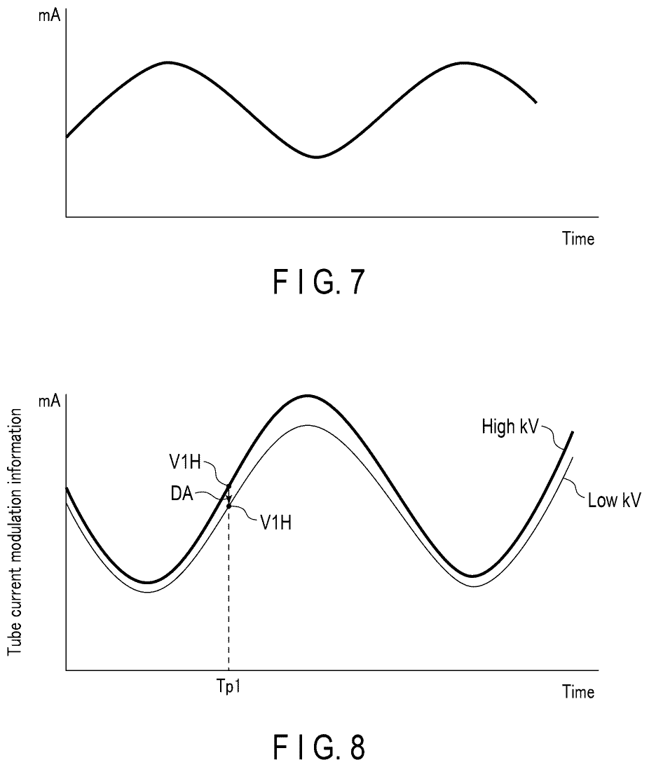

FIG. 7 is a diagram illustrating, as an example, tube current modulation information for reference tube voltage application (high tube voltage application). In FIG. 7, the vertical axis denotes a tube current value mA, and the lateral axis denotes time. The time refers to a period of time that has passed since the starting time of X-ray irradiation, and corresponds to an X-ray tube angle. FIG. 7 shows tube current modulation information for a single rotation of the X-ray tube. The tube current modulation information for reference tube voltage application represents a change in the tube current setting value in accordance with a change in time at the time of application of a high tube voltage. The tube current value at each time point is determined based on, for example, an X-ray tube angle at that time and a subject thickness (more specifically, a water equivalent thickness) of an X-ray path at the X-ray tube angle. The subject thickness may be measured by subjecting a positioning image to image processing, or by means of an optical camera, laser, or the like, or may be input by the user via the input interface 43. The processing circuitry 45 determines a subject thickness at each time point, and determines a setting tube current value in accordance with a known algorithm based on the determined subject thickness and an image quality evaluation value. For the image quality evaluation value, a signal-to-noise ratio (SNR), a contrast-to-noise ratio (CNR), or the like is adopted. It is preferable, for example, that the setting tube current value be set in such a manner that an even image quality is obtained at varying X-ray tube angles, while minimizing the radiation amount.

After step SB1, the processing circuitry 45 reads the tube current characteristic data (step SB2). The tube current characteristic data associated with a combination of a high tube voltage value and a low tube voltage value used in tube current modulation spectrum scanning to be performed is read from the memory 41.

After step SB2, the processing circuitry 45 generates, with the scanning condition setting function 51, tube current modulation information for low tube voltage application based on the tube current modulation information for high tube voltage application, using the tube current characteristic data read at step SB2 (step SB3).

FIG. 8 is a diagram illustrating tube current modulation information (High kV) for high tube voltage application and tube current modulation information (Low kV) for low tube voltage application. In FIG. 8, the vertical axis denotes a tube current value mA, and the lateral axis denotes time. The time refers to a period of time that has passed since the starting time of X-ray irradiation, and corresponds to an X-ray tube angle. The processing circuitry 45 specifies, based on the tube current characteristic data, a tube current fluctuation amount representing a fluctuation amount of a tube current output value under an identical filament current value, for each point of the tube current modulation information for high tube voltage application, and calculates a tube current setting value for low tube voltage application based on the tube current setting value for high tube voltage application and the tube current fluctuation amount, thereby generating tube current modulation information for low tube voltage application.

First, a time point to be processed is set. The time point to be processed is sequentially set for all time points forming the tube current modulation information. It is assumed, as an example, that a time point Tp1 is set. First, a tube current setting value V1H at the time point Tp1 is specified based on the tube current modulation information for high tube voltage application.

Subsequently, the same tube current value V1H is specified based on a tube current characteristic curve of the high tube voltage of the tube current characteristic data concerning a combination of the same high tube voltage and a low tube voltage, and a tube current fluctuation amount DA at the time of the specified tube current value V1H is specified. By subtracting the specified tube current fluctuation amount DA from the tube current value V1H, a tube current setting value V1L for low tube voltage application is determined. If, for example, the tube current value at the time point Tp1 is Va1H, a tube current fluctuation amount da1 is specified based on the tube current characteristic curve of the high tube voltage of the tube current characteristic data shown in FIG. 5, and the tube current fluctuation amount da1 is subtracted from the tube current setting value Va1H, and thereby a tube current setting value Va1L for low tube voltage application is determined. By executing the above-described processing at each time point, it is possible to generate tube current modulation information for low tube voltage application.

As described above, the tube current characteristic data is generated based on a tube current value obtained in the filament heated by performing, in the X-ray tube 11 used for tube current modulation spectrum scanning, kV switching between a high tube voltage value and a low tube voltage value to be actually adopted. Accordingly, by generating a tube current characteristic curve of the low tube voltage based on a tube current characteristic curve of the high tube voltage via the tube current characteristic data, it is possible, at each time point, to obtain a tube current setting value for high tube voltage application and a tube current setting value for low tube voltage application at an identical filament temperature. By alternately setting the tube current setting value from the tube current characteristic curve of the high tube voltage and from the tube current characteristic curve of the low tube voltage thus obtained, continuity of the filament temperature can be ensured over the duration of the kV switching, and it can be expected that the tube current feedback control is accurately performed at the time of both the high tube voltage application and the low tube voltage application.

After step SB3, the processing circuitry 45 performs, with the scanning control function 52, tube current modulation spectrum scanning (step SB4). At step SB4, the processing circuitry 45 performs tube current feedback control at the time of both the high tube voltage application and the low tube voltage application by switching the tube current setting value based on the tube current modulation information for high tube voltage application generated at step SB1 and the tube current modulation information for low tube voltage application generated at step SB3, in synchronization with the switching between the high tube voltage and the low tube voltage.

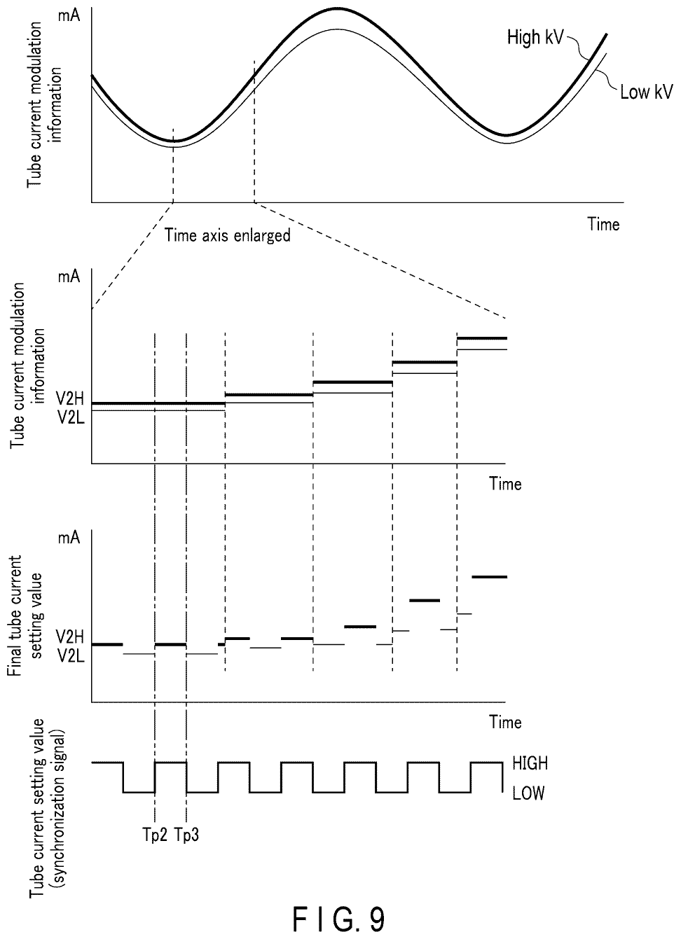

FIG. 9 is a diagram showing a control sequence diagram of tube current modulation spectrum scanning. As shown in the first row of FIG. 9, tube current modulation information for high tube voltage application is acquired at step SB1, and tube current modulation information for low tube voltage application is acquired at step SB3. In the second row of FIG. 9, the time axis is partially enlarged, considering that the kV switching is performed at high speed relative to such a change. In actuality, the setting tube current value is discretely set, as shown in the second row of FIG. 9. The tube current value can be switched multiple times during the period of a single application of the high tube voltage or the low tube voltage.

The third row of FIG. 9 denotes a transition of a final tube current setting value, and the fourth row of FIG. 9 denotes a transition of the tube voltage setting value. As shown in the fourth row of FIG. 9, in tube current modulation spectrum scanning, the tube voltage control circuitry 72 performs kV switching by performing tube voltage feedback control by alternately switching between the high tube voltage setting value and the low tube voltage setting value in accordance with a synchronization signal. The synchronization signal is a signal that is repeatedly brought to a HIGH level and a LOW level every time the X-ray tube is rotated by a certain angle. The HIGH level corresponds to the high tube voltage application period, and the LOW level corresponds to the low tube voltage application period. The synchronization signal is supplied from the controller 15, etc.

The tube current control circuitry 77 performs tube current feedback control at the time of both the high tube voltage application and the low tube voltage application by alternately switching, in accordance with the synchronization signal, the tube current setting value based on the tube current modulation information for high tube voltage application and the tube current modulation information for low tube voltage application, in parallel with the kV switching. Specifically, the tube current setting circuitry 81 tracks the current time in both the tube current modulation information for high tube voltage application and the tube current modulation information for low tube voltage application. Triggered by the synchronization signal switched from LOW to HIGH, the tube current setting circuitry 81 specifies the tube current setting value at the current time based on the tube current modulation information for high tube voltage application, and sets the specified tube current setting value as a final tube current setting value. The tube current setting signal corresponding to the final tube current setting value is supplied to the tube current comparison circuitry 80. The tube current comparison circuitry 80 outputs a differential tube current signal between the tube current setting signal and the tube current output signal.

The tube current control circuitry 77 controls the tube current in such a manner that the differential tube current signal converges to zero, in other words, the tube current output value converges to the final tube current setting value. Thereby, tube current feedback control is performed at the time of the high tube voltage application.

Similarly, triggered by the synchronization signal switched from HIGH to LOW, the tube current setting circuitry 81 specifies the tube current setting value at the current time based on the tube current modulation information for low tube voltage application, and sets the specified tube current setting value as a final tube current setting value. The tube current setting signal corresponding to the final tube current setting value is supplied to the tube current comparison circuitry 80. The tube current comparison circuitry 80 outputs a differential tube current signal between the tube current setting signal and the tube current output signal, and the tube current control circuitry 77 controls the tube current in such a manner that the differential tube current signal converges to zero, in other words, the tube current output value converges to the final tube current setting value. Thereby, tube current feedback control is performed at the time of the low tube voltage application.

At time Tp2 at which the synchronization signal is switched from LOW to HIGH, for example, a tube current setting value V2H is set as a final tube current setting value from the tube current modulation information for high tube voltage application, and tube current feedback control is performed based on the tube current setting value V2H at the time of the high tube voltage application. At time Tp3 at which the synchronization signal is switched from HIGH to LOW, a tube current setting value V2L is set as a final tube current setting value from the tube current modulation information for low tube voltage application, and tube current feedback control is performed based on the tube current setting value V2L at the time of the low tube voltage application.

As described above, according to the present embodiment, since the continuity of the filament temperature can be ensured over the duration of the kV switching, it is possible to accurately perform tube current feedback control at the time of both the high tube voltage application and the low tube voltage application.

With the foregoing, processing of the tube current modulation spectrum scanning ends.

The above-described embodiment is merely an example and is not limited thereto, and deletion, addition, and/or modification of various elements may be made as along as the gist of the invention is not altered.

For example, in the embodiment shown in FIG. 6, tube current modulation information for low tube voltage application is generated in advance prior to performing tube current modulation spectrum scanning, and a tube current setting value is set based on the tube current modulation information for low tube voltage application during scanning at the time of the low tube voltage application. However, the present embodiment is not limited thereto. Instead of generating the tube current modulation information for low tube voltage application, a tube current setting value for the low tube voltage application may be determined and set in real time during scanning based on tube current modulation information for high tube voltage application and tube current characteristic data. Specifically, it suffices that the tube current setting circuitry 81 tracks the current time in both the tube current modulation information for high tube voltage application and the tube current modulation information for low tube voltage application, specifies, triggered by switching of a synchronization signal from HIGH to LOW, a tube current value at the current time based on the tube current modulation information for high tube voltage application, specifies a tube current fluctuation amount corresponding to the specified tube current value based on tube current characteristic data, and sets a value obtained by subtracting the specified tube current fluctuation amount from the tube current value as a tube current setting value.

It can be expected that the characteristics of the X-ray tube 11 change with prolonged use. If the filament deteriorates after prolonged use, the fluctuation of the filament current increases at the time of kV switching. Thus, the processing circuitry 45 determines, with the re-acquisition determination function 57, whether or not the tube current characteristic data needs to be re-acquired based on an amount of fluctuation of the filament current in accordance with the switching between the high tube voltage and the low tube voltage. With the display control function 58, the processing circuitry 45 displays a determination result as to whether or not the tube current characteristic data needs to be re-acquired.

The filament current detection circuitry 83 records the amount of fluctuation of the filament current every time kV switching is performed, for example. Specifically, an amount of fluctuation of the filament current in accordance with the switching from the high tube voltage application to the low tube voltage application or from the low tube voltage application to the high tube voltage application is recorded in the memory 41, the memory 82, or the like. Note that kV switching may be performed at the time of tube current modulation spectrum scanning, or at the time of a tube current fluctuation amount measurement mode.

The processing circuitry 45 compares the amount of fluctuation of a filament current to be determined with a permissible value. The amount of fluctuation of the filament current to be determined is freely set from fluctuation amounts recorded in the memory 41, the memory 82, or the like. As the permissible value, a value that separates fluctuation amounts measured in a deteriorated filament from fluctuation amounts measured in a non-deteriorated filament may be set, or a value that is representative of the fluctuation amounts measured in a deteriorated filament may be set. As the permissible value, a value obtained by statistical analysis or the like may be set, or a value freely determined by the user or the like may be set. If the amount of fluctuation of the filament current to be determined is greater than the permissible value, the processing circuitry 45 determines that the tube current characteristic data needs to be re-acquired. On the other hand, if the amount of fluctuation of the filament current to be determined is smaller than the permissible value, the processing circuitry 45 determines that the tube current characteristic data does not need to be re-acquired. A result of the determination as to whether or not the tube current characteristic data needs to be re-acquired is displayed on the display 42 in a predetermined layout.

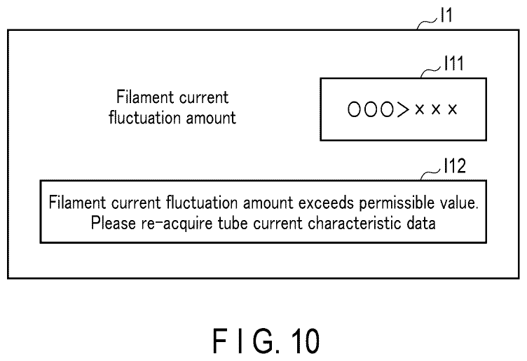

FIG. 10 is a diagram illustrating a display screen I1 showing a result of determination as to whether or not the tube current characteristic data needs to be re-acquired. As shown in FIG. 10, the display screen I1 includes a display column I11 and a display column I12. In the display column I11, a comparison such as “OOO>XXX” between the amount of fluctuation of the filament current (“OOO”) and the permissible value (“XXX”) is displayed. In the display column I12, a message indicating whether or not the tube current characteristic data needs to be re-acquired is displayed. If, for example, the amount of fluctuation of the filament current to be determined is greater than the permissible value and it has been determined that the tube current characteristic data needs to be re-acquired, a message such as “Filament current fluctuation amount exceeds permissible value. Please re-acquire tube current characteristic data.” is displayed.

If it has been determined that the tube current characteristic data needs to be re-acquired, the tube current fluctuation amount measurement mode shown in FIG. 3 is performed, and the tube current characteristic data is re-acquired. By thus monitoring the amount of fluctuation of the filament current, it is possible to determine whether or not the tube current characteristic data needs to be re-acquired. By re-acquiring the tube current characteristic data in accordance with deterioration of the filament, it is possible to perform tube current feedback control based on tube current characteristic data that conforms to the behavior of the filament current or the actual filament temperature, thereby improving the precision of the tube current modulation.

According to at least some of the embodiments described above, the X-ray computed tomography imaging apparatus 1 includes an X-ray tube 11, a memory 82, tube voltage control circuitry 72, tube current setting circuitry 81, and tube current control circuitry 77. The X-ray tube 11 is configured to generate X-rays. The memory 82 is configured to store tube current characteristic data representing a relationship between a filament current value and a tube current output value in a case of switching of the tube voltage to be applied to the X-ray tube 11 between a first tube voltage and a second tube voltage lower than the first tube voltage. The tube voltage control circuitry 72 is configured to switch the tube voltage to be applied to the X-ray tube 11 between the first tube voltage and the second tube voltage. The tube current setting circuitry 81 is configured to set a tube current setting value for performing tube current modulation in parallel with switching between the first tube voltage and the second tube voltage, in such a manner that a first tube current setting value is set at a time of application of either the first tube voltage or the second tube voltage whichever is a reference tube voltage, and a second tube current setting value based on tube current characteristic data and a first tube current setting value is set at a time of application of the other one of the first tube voltage or the second tube voltage. Tube current control circuitry 77 is configured to perform tube current feedback control based on the first tube current setting value at the time of application of the reference tube voltage, and perform second tube current feedback control based on the second tube current setting value at the time of application of the other tube voltage, in synchronization with the switching between the first tube voltage and the second tube voltage.

According to the above-described configuration, the tube current characteristic data is generated based on a tube current value obtained in the filament heated by actually performing, in the X-ray tube 11 used for tube current modulation spectrum scanning, kV switching between a high tube voltage value and a low tube voltage value that may be actually adopted. It is thus possible to secure the continuity of the filament temperature at the time of both the first tube voltage application and the second tube voltage application based on the tube current characteristic data and the first tube current setting value at the time of application of the reference tube voltage. It is thereby possible to suppress fluctuation of the filament current over the duration of the kV switching, and it can be expected that both the tube current feedback control based on the first tube current setting value and the tube current feedback control based on the second tube current setting value can be accurately performed.

There is a control scheme (hereinafter referred to as a “comparative example scheme”) in which tube current feedback control is performed at the time of application of a reference tube voltage, and tube current feedback control is not performed at the time of application of the other tube voltage. In the comparative example scheme, since the tube current feedback control is alternately switched between ON and OFF, the filament current greatly fluctuates at the time of the switching. To suppress the fluctuation of the filament current, a difference (a tube current fluctuation amount) between the maximum tube current value and the minimum tube current value in the tube current modulation must be decreased, and the radiation dose cannot be reduced as desired.

According to the present embodiment, since tube current feedback of both the reference tube voltage and the other tube voltage can be performed while suppressing fluctuation of the filament current, it is possible to increase the tube current fluctuation amount compared to the comparative example scheme, thus reducing the radiation dose compared to the comparative example scheme.

According to at least one embodiment described above, it is possible to improve the precision, responsibility, and permissible modulation width of the tube current modulation at the time of kV switching.

The term “processor” used in the above explanation means, for example, circuitry such as a central processing unit (CPU), a graphics processing unit (GPU), an application-specific integrated circuit (ASIC), or a programmable logic device (e.g., a simple programmable logic device (SPLD), a complex programmable logic device (CPLD), or a field programmable gate array (FPGA)). The processor reads a program stored in storage circuitry and executes the program to execute the corresponding function. Note that a program may be directly incorporated in circuitry of the processor instead of being saved in storage circuitry. In this case, the processor reads the program incorporated in the circuitry and reads and executes the program to implement the corresponding function. On the other hand, if the processor is, for example, an ASIC, the corresponding function is directly incorporated as logic circuitry in the circuitry of the processor instead of the program being saved in the storage circuit. Each processor of the present embodiment is not necessarily configured as a single circuit, and a plurality of independent circuits may be combined into a single processor to realize the respective functions. In addition, a plurality of constituent elements shown in FIGS. 1 and 2 may be integrated into a single processor to implement the corresponding function.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray computed tomography imaging apparatus, comprising:
an X-ray tube configured to generate X-rays;
a memory configured to store characteristic data representing a relationship between a filament current value and a tube current output value in a case of switching of a tube voltage to be applied to the X-ray tube between a first tube voltage and a second tube voltage lower than the first tube voltage;
tube voltage control circuitry configured to switch the tube voltage to be applied to the X-ray tube between the first tube voltage and the second tube voltage;
tube current setting circuitry configured to set a tube current setting value for performing tube current modulation in parallel with switching between the first tube voltage and the second tube voltage, the tube current setting circuitry being configured to set a first tube current setting value at a time of application of either the first tube voltage or the second tube voltage, whichever is a reference tube voltage, and to set a second tube current setting value based on the characteristic data and the first tube current setting value at a time of application of the other one of the first tube voltage or the second tube voltage; and
tube current control circuitry configured to perform tube current feedback control based on the first tube current setting value at a time of application of the reference tube voltage, and to perform tube current feedback control based on the second tube current setting value at a time of application of said other tube voltage, in synchronization with the switching between the first tube voltage and the second tube voltage.

2. The X-ray computed tomography imaging apparatus according to claim 1, further comprising:
processing circuitry configured to generate first tube current modulation information indicating a temporal change in the first tube current setting value for periodically modulating a tube current, and to generate second tube current modulation information indicating a temporal change in the second tube current setting value based on the first tube current modulation information and the characteristic data, wherein
the processing circuitry sets a present value of the first tube current modulation information at the time of application of the reference tube voltage as the first tube current setting value, and sets a present value of the second tube current modulation information at the time of application of said other tube voltage as the second tube current setting value.

3. The X-ray computed tomography imaging apparatus according to claim 2, wherein
the processing circuitry generates the second tube current modulation information by specifying, based on the characteristic data, a tube current fluctuation amount for each point of the first tube current modulation information, and calculates the second tube current setting value based on the first tube current setting value and the tube current fluctuation amount, the tube current fluctuation amount being a fluctuation amount of a tube current output value under an identical filament current value.

4. The X-ray computed tomography imaging apparatus according to claim 2, wherein
the processing circuitry calculates the first tube current modulation information based on a body thickness of a subject and an image quality evaluation value.

5. The X-ray computed tomography imaging apparatus according to claim 1, further comprising processing circuitry configured to:
measure, for each of a plurality of filament current values, a tube current output value of the X-ray tube and a tube current fluctuation amount in a case of switching a tube voltage between the first tube voltage and the second tube voltage, with the filament current value fixed, the tube current fluctuation amount being a fluctuation amount of the tube current output value; and
generate the characteristic data for a combination of the first tube voltage and the second tube voltage based on the plurality of filament current values and the tube current output value and the tube current fluctuation amount for each of the plurality of filament current values.

6. The X-ray computed tomography imaging apparatus according to claim 5, wherein
the processing circuitry is configured to measure the tube current output value and the tube current fluctuation amount in a period of stabilization of a temperature of the filament included in the X-ray tube.

7. The X-ray computed tomography imaging apparatus according to claim 1, further comprising:
filament current detection circuitry configured to measure a filament current flowing through a filament included in the X-ray tube; and
processing circuitry configured to determine whether or not the characteristic data needs to be re-acquired, based on an amount of fluctuation of the filament current in accordance with the switching between the first tube voltage and the second tube voltage, and to display a result of the determining as to whether or not the characteristic data needs to be re-acquired.

8. The X-ray computed tomography imaging apparatus according to claim 1, wherein
the reference tube voltage is the first tube voltage.

9. An X-ray high-voltage apparatus, comprising:
a memory configured to store characteristic data representing a relationship between a filament current value and a tube current output value in a case of switching of the tube voltage to be applied to an X-ray tube between a first tube voltage and a second tube voltage lower than the first tube voltage;
tube voltage control circuitry configured to switch the tube voltage to be applied to the X-ray tube between the first tube voltage and the second tube voltage;
tube current setting circuitry configured to set a tube current setting value for periodically modulating a tube current of the X-ray tube in parallel with the switching between the first tube voltage and the second tube voltage, the tube current setting circuitry being configured to set a first tube current setting value at a time of application of either the first tube voltage or the second tube voltage, whichever is a reference tube voltage, and to set a second tube current setting value based on the characteristic data and the first tube current setting value at a time of application of the other one of the first tube voltage or the second tube voltage; and
tube current control circuitry configured to switch between tube current feedback control based on the first tube current setting value at the time of application of the reference tube voltage, and tube current feedback control based on the second tube current setting value at the time of application of said other tube voltage, in synchronization with switching between the first tube voltage and the second tube voltage.

10. An X-ray control method, comprising:
a step of switching a tube voltage to be applied to an X-ray tube between a first tube voltage and a second tube voltage lower than the first tube voltage;
a step of setting a tube current setting value for periodically modulating a tube current of the X-ray tube in parallel with switching between the first tube voltage and the second tube voltage, in such a manner that a first tube current setting value is set at a time of application of either the first tube voltage or the second tube voltage, whichever is a reference tube voltage, and a second tube current setting value based on characteristic data and the first tube current setting value is set at a time of application of the other one of the first tube voltage or the second tube voltage, the characteristic data representing a relationship between a filament current value and a tube current output value in a case of switching of the tube voltage to be applied to the X-ray tube between the first tube voltage and the second tube voltage; and
a step of switching between tube current feedback control based on the first tube current setting value at the time of application of the reference tube voltage, and tube current feedback control based on the second tube current setting value at the time of application of said other tube voltage in synchronization with the switching between the first tube voltage and the second tube voltage.
